(19) United States
(12) Patent Application Publication
(10) Pub. No.: US 2014/0363484 A1
(43) Pub. Date: Dec. 11, 2014

(54) FIBROUS BIO-DEGRADABLE POLYMERIC WAFERS SYSTEM FOR THE LOCAL DELIVERY OF THERAPEUTIC AGENTS IN COMBINATIONS

(71) Applicants: MANZOOR KOYAKUTTY, Kochi (IN); RANJITH RAMACHANDRAN, Kochi (IN); SHANTIKUMAR NAIR, Kochi (IN)

(72) Inventors: MANZOOR KOYAKUTTY, Kochi (IN); RANJITH RAMACHANDRAN, Kochi (IN); SHANTIKUMAR NAIR, Kochi (IN)

(73) Assignee: Amrita Vishwa Vidyapeetham, Kochi (IN)

(21) Appl. No.: 14/465,642

(22) Filed: Aug. 21, 2014

Related U.S. Application Data
(63) Continuation of application No. PCT/IN2013/000110, filed on Feb. 20, 2013.

(30) Foreign Application Priority Data
Feb. 21, 2012 (IN) 643/CHE/2012

(51) Int. Cl. A61K 9/70; A61K 47/34

(52) U.S. Cl. CPC A61K 9/70; A61K 47/34
USPC 424/443

(57) ABSTRACT

The present invention is related to flexible, fibrous, biocompatible and biodegradable polymeric wafer; consists of more than one polymeric fibers, each one loaded with different therapeutic agents having mutually exclusive synergistic activity. The wafer is capable of delivering the drugs locally in to the diseased site like tumor, inflammation, wound, etc., in a controlled and sustained fashion for enhanced therapeutic effect. The combination of drugs loaded in the wafer is chosen in such a way that the second or consecutive drugs will enhance or improve the therapeutic effect of the first drug.

### CROSS-REFERENCES TO RELATED APPLICATIONS

This application is a continuation of PCT international application PCT/IN2013/000110 filed on 20 Feb. 2013, which claims priority to Indian patent application No. 643/CHE/2012, filed on 21 Feb. 2012, the full disclosure of which is incorporated herein by reference.

### FIELD OF THE INVENTION

The invention is related to fibrous, flexible, biodegradable and biocompatible polymeric wafer system for the local delivery of therapeutic agents in combination. More particularly, the system intended for the delivery of combination of therapeutic agents, for example anti-neoplastic drugs, locally to the diseased site in a controlled and sustained fashion.

### BACKGROUND

Current methods for drug delivery have very limited utility due to their inability to deliver drugs locally to a specific organ or tissue in clinically significant doses. Most conventional drug delivery methods can only allow a small concentration of the drug to reach a specific target location because of wide drug distribution, high plasma-protein binding, low bio-availability and short half-life. Also, most chemotherapeutics being hydrophobic in nature will tend to form aggregates and get cleared from the circulation very fast. Most of the anti-neoplastic agents need to be administered repeatedly in high systemic concentrations for effective therapy causing toxicities even to the normal cells. Most of these problems can be effectively addressed by using local drug delivery devices that will provide a sustained and controlled delivery of drugs delivery in desired fashion.

Local drug delivery is difficult in case of highly isolated organs such as brain where the blood-brain barrier is an additional factor. Central nervous system tumors, for example glioblastoma (GBM) are diseases that would benefit from local drug delivery. The standard of care therapy for GBM is surgical resection of the tumor followed by chemotherapy and/or radiation therapy. Complexity in complete tumor resection and limitations faced by supporting therapies make its cure very difficult and the median survival rate still remains less than 12 months. Despite the remarkable increase in the number of anti-cancer drugs discovered, chemotherapy for CNS tumors still faces apparent ineffectiveness due to the unique environment of the brain. Brain, being an important and delicate organ, is protected by specialized mechanisms; the most important one being the blood brain barrier (BBB). This barrier significantly reduces the permeability of capillary walls to effectively block large molecules and peptides from getting into the brain. In addition the blood cerebrospinal fluid barrier and blood-tumor barrier also work to reduce the permeation of drug molecules into the required areas in the brain. Hence only molecules having very small size (<400 Da) that are electrically neutral and lipid soluble can easily pass through it, and most chemotherapeutic agents are excluded from this category. Even in the case of small drug molecules which have limited permeability through the BBB, achieving a clinically significant concentration locally at the tumor site for effectiveness of chemotherapy necessitates administration of very high systemic doses. This can lead to systemic toxicities and other adverse drug events, necessitating dosage limitations and ultimately causing treatment failure. Most of the potent chemotherapeutic drugs like DNA alkylating/intercalating agents, anti-angiogenic agents, cytokines, small molecule inhibitors, DNA alkylating agents etc. fall in this category.

In order to circumvent these problems regarding brain drug delivery, various strategies have been developed by researchers, like changing the drug design for increased permeability, disrupting the BBB temporarily, localized drug delivery etc. In these methods, local drug delivery using biocompatible polymeric devices or microchips is one of the most important therapeutic strategies that shows promising outcome in cancer management. Gliadel® was the first locally implantable polymeric drug delivery device approved by FDA. Gliadel® is made of pCPP-SA (poly[bis(p-carboxyphenoxy)propane-co-sebacic acid] polymer incorporating 3.8% wt/wt carmustine (BCNU; 1,3-bis(2-chloroethyl)-1-nitroso-urea) and provides an effect means of its direct delivery. These devices are implanted into the cavity resulting from the surgical resection of the tumor. Gliadel® thus can provide sustained release of BCNU approximately up to 3 weeks and has shown effectiveness to improve patient survival significantly. This local chemotherapy can be used along with other conventional therapies like radiotherapy without causing any limitations to them. Although Gliadel® therapy provides benefits to cancer management, limitations such as its extreme brittle nature, handling difficulties and inability to provide extended sustained release limit its usefulness.

Recent studies also prove that tumors develop different mechanisms for drug efflux, ROS scavenging, DNA repair etc. to prevent or overcome the damage caused by chemotherapy. In order to avoid these limitations combinatorial therapeutic approaches were introduced, which combine conventional chemotherapeutic agents with drugs that inhibit the cell's drug resistance mechanisms. For example temozolomide, a potent chemotherapeutic drug, acts by alkylating DNA bases mostly in O6 position of guanine residue. These altered bases will cause mispairing during DNA replication, leading to DNA repair associated cell death. But, cancer cells (e.g., glioma) over express MGMT protein that can remove these alkyl groups and help the cells survive. A clinically accepted combination therapy to such cancer uses O6-benzyl guanine, a substrate analogue that irreversibly inhibits MGMT enzyme, thereby making the cells sensitive to temozolomide. Success of such a combinatorial approach primarily depends on achieving clinically significant concentrations of both drugs locally at the tumor site in a desired fashion. For example, in this case the therapy will be highly efficient if O6-BG is applied just before TMZ administration. Also temozolomide, being very short-lived (half-life is 1.8 h) under physiological conditions, when administered repeatedly in high doses for desired treatment effect causes significant systemic toxicities and related adverse drug-effects.

This can be overcome by delivering these drugs locally at the tumor site using drug delivery wafers. Success of such a drug delivery device depends on many factors including stability of the drugs loaded, drug-loading efficiency, achieving sustained release with desirable release kinetics etc. Gliadel® like device made of simple incorporation of these drugs cannot achieve these properties necessary for the combinatorial treatment approach. Also clinicians face difficulty with their highly-brittle and non-flexible nature. These factors demand a flexible device that can deliver the combination of drugs in derided and sustained fashion for optimal treatment efficacy. The emerging field of nanotechnology offers great promise for such drug delivery applications. For example, in the above mentioned case, the optimum drug deliveries can be achieved by electrospun/rotary jet-spun wafers with flexible nature and tenable degradation kinetics.

Even though many local drug delivery (or drug eluting) devices have been developed world wide to treat diseases like cancer, especially brain cancers, there exist a very few devices made of biodegradable polymers giving a sustained drug release. These devices are made by mixing the drug (3.8% wt/wt) with pCPP-SA polymers and making discs by applying pressure pelletizing and aid release up to 3 weeks. Another polymeric drug delivery device is DC bead®. It is produced from biocompatible polyvinyl alcohol (PVA) hydrogel that has been modified with sulphonate groups for the controlled loading and delivery of chemotherapeutic drugs and in trans-arterial chemoembolisation. They occlude the blood flow to the target tissue and deliver a local and sustained dose of the loaded drug (e.g. doxorubicin, irinotecan, etc.) direct to the tumor.

Even though various local drug delivery devices were prepared and studied by the researchers worldwide, there is only little literature available which deals with drug delivery using electrospun or rotary jet-spinning method. Also, most of the literature deals with delivery of a single chemotherapeutic agent. Liao et al have studied the Preparation, characterization, and encapsulation/release studies of a composite nanofiber mat electrospun from an emulsion containing PLGA. Ranganath et al have studied Paclitaxel-loading in biodegradable electrospun polymeric implants in the form of microfiber discs and sheets and investigated its efficiency against malignant glioma. They fabricated wafers of PLGA fibers having submicron size diameter loaded with pacletaxel. Xie et al also studied pacletaxel loading in electrospun PLGA fibers and its effects on C6 Glioma both in vitro and in vivo. He also studied PLA/PLGA electrospun fibers for local delivery of cisplatin.

Even though biodegradable polymeric fibrous or electrospun devices were used in few of the prior arts for localized delivery of single or multiple therapeutic agents, fibrous wafers made up of two different kinds of polymeric fibers loaded separately with two different drugs capable of releasing the two in a controlled and sustained fashion for >1 month for enhanced combinatorial approach are not reported. Furthermore, in our method, the polymers and solvent used are chosen critically for the optimal loading, stability, sustained release of the encapsulated molecules and required release kinetics for combinatorial chemotherapy.

Accordingly, there exist a need for an use of flexible, handy, fibrous, biodegradable and biocompatible polymeric wafers consists of more than one type of polymeric fibers, each loaded separately with different therapeutic agent aiding combination therapy and also capable of delivering the drug in a controlled and sustained fashion for one week and up to many months, locally in to or to the vicinity of the diseased area or tissue for local drug delivery applications. Eyen in prior arts detailing drug delivery wafers with two or more different polymer fibers loaded with drugs, there exist no suggestion obtaining the optimum release kinetics needed for the combination therapy, and also there is no suggestion in criteria for choosing the drug-combinations for synergistic therapeutic effects because of mutually exclusive activity.

### SUMMARY OF THE INVENTION

A flexible and biodegradable wafer system for delivering multiple therapeutic agents is disclosed. In one aspect the system comprises first and second polymeric fibers and plurality of therapeutic agents. In one aspect, the first and second polymeric fiber configured as a flexible fibrous wafer loaded with therapeutic agents. In various aspects, the therapeutic agents comprises with mutually exclusive synergistic activity. In various aspects, fibrous wafer to provide a combined therapy with sustained and controlled release of the therapeutic agents in the diseased site. In one aspect, the polymer fibers have an average diameter between 1-50,000 nm. In one aspect, the polymer fibers are porous or non-porous, beaded or non-beaded, uniform or non-uniform, solid or hollow, or ribbon-shape in nature. In one aspect, the first and second polymer fibers possess different release kinetics. In various aspects, the drugs loaded in the fibers are in their pure molecule form or in their slated form or in their nano-encapsulated form. In various aspects, the fibers are randomly oriented fibers. In various aspects, the fibers are aligned fibers.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 shows fibrous bio-degradable polymeric wafers system.

FIG. 2 shows different steps involved in wafer making through electrospinning method.

FIGS. 3A-3F show SEM images for different morphology of polymeric fibers obtained electrospinning technique.

FIG. 4 shows an SEM image for microscopic fiber morphology of TMZ and O6-BG co-loaded PLA-PLGA/PLGA wafers.

FIGS. 5A-5C show EDS mapping results with uniform distribution of drugs throughout polymeric fibers.

FIG. 6 shows FTIR results with interaction of TMZ with PLA-PLGA blend polymeric matrix.

FIG. 7 shows a graph representing near-zero order temozolomide release.

FIG. 8 shows a graph representing near-zero order O6-Benzylguanine release.

FIGS. 9A-D represent cell attachment studies of bare and drug-loaded wafers showing effective inhibition of cell attachment and proliferation by the drug loaded wafers.

FIG. 10 represents in vitro live-dead assay results showing effective cell growth inhibition by the drug loaded wafers. Cells were seen live and attached (in green fluorescence, due to esterase activity) in the bare wafers (Upper panel) whereas no cells were attached onto drug-loaded wafers (Down panel).

### DETAILED DESCRIPTION OF THE PREFERRED EMBODIMENTS

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The term “polymeric fibers” as used herein refers to fibers formed by electrospraying or rotary jet-spinning of polymer solution. Fibers may have a diameter of about 10 nm-50,000 nm. In one embodiment the fibers have a diameter of about 10-1000 nm. In another embodiment the fibers have a diameter of around 1-250 nm in size.

The terms “biodegradable” refers to the degradation or disassembly of a polymer by action of a biological environment by the way of linkage breakdown by mechanisms such as hydrolysis, enzyme, pH or temperature degradation.

The term “loading” as used herein refers to uniform or non-uniform incorporation of monomeric or aggregated forms of the therapeutic agent inside or outside or throughout or though the surface of the polymer fibers.

The term “chemotherapeutic agent” or “chemotherapeutic drug” as used herein are similar and refers to compounds or molecules which produces a beneficial or useful for cancer treatment.

The terms “controlled release”, “sustained release” and similar terms are used to denote a mode of delivery of the therapeutic agent that occurs when the agent is released from the polymeric wafer at an ascertainable and manipulatable rate over a period of time, rather than dispersed immediately upon application. Controlled or sustained release may extend for hours, days or months, and may vary as a function of numerous factors. An important determinant of the rate of delivery is the rate of hydrolysis of the linkages between and within the units of the polymer. The rate of hydrolysis in turn may be controlled by the factors like the composition of the wafer, polymer used, its molecular weight, monomer ratios, hydrophilicity, fiber diameter, presence and absence of beads, fiber porosity etc. Other factors include implant size, length of the electro spun fibers, acidity of the medium, solubility of the active agent in the matrix, molecular weight and charge density of the active ingredient.

The term mutually exclusive synergistic activity means the therapeutic effect by the combination of drugs are enhanced or much better than that of individual drugs as the activity of one drug helps to improve the effect of another drug.

The present disclosure relates to fibrous bio-degradable polymeric wafers system for the local delivery of therapeutic agents in combinations is described in the following sections referring to the sequentially numbered figures. In one aspect, the fibrous bio-degradable polymeric wafers system is configured to be specifically targeted to the preferred site of action and configured to controllably release therapeutic agents.

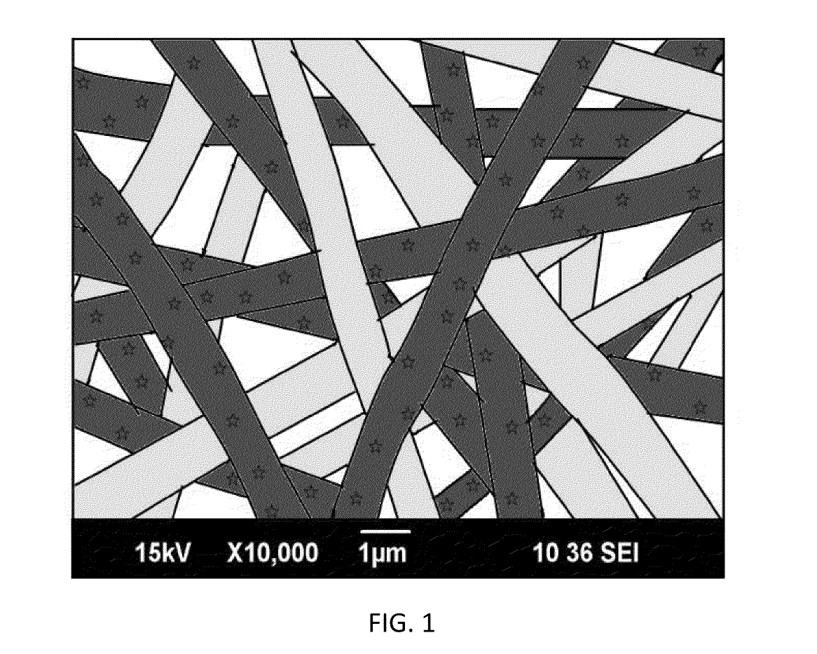

In one embodiment, fibrous bio-degradable polymeric wafers system for the local delivery of therapeutic agents is disclosed, as shown in FIG. 1. As shown in FIG. 1, in one embodiment, the system comprises first 101 and second 102 polymeric fibers, and plurality of therapeutic agents 103. In one embodiment, the first 101 and second 102 polymeric fiber configured as a flexible fibrous wafer 104 loaded with therapeutic agents 103. The therapeutic agents 103 comprises with mutually exclusive synergistic activity. In one embodiment, fibrous wafer 104 is configured to provide a combined therapy with sustained and controlled release of the therapeutic agents 103 in the diseased site.

In one embodiment of the said wafer 104, wherein the different degradation kinetics for each kind of fibers is achieved by using polymers or polymer blend with differed degradation or by using same polymers with different molecular weight or by using same polymers with altered monomer ratio. For example PLGA (85:15) will have extended degradation than that of PLGA (50:50). Also a polymer with higher molecular weight will degrade slow compared to same polymer with a lower molecular weight. The degradation of polymers will depend on factors such as the rate of hydrolysis of the linkages between and within the units of the polymer. The rate of hydrolysis in turn may be controlled by the factors like the compositions of the wafer, polymer used, its molecular weight, monomer ratios, hydrophilicity, fiber diameter, presence and absence of beads, fiber porosity etc. Other factors include implant size, length of the electro spun fibers, and acidity of the medium, solubility of the active agent in the matrix, molecular weight and charge density of the active agent.

In one embodiment of said fibrous wafer 104, the polymer fibers are formed by a known method chosen from electrospinning or rotary jet spinning in co-spinning, sequential spinning, simultaneous spinning fashion as specified for the optimal release of the incorporated drugs.

In various embodiments, fibers 101 and 102 are natural or synthetic biocompatible polymer at least one from the group, but not limited to poly glycolic acid (PGA), poly(lactic-co-glycolic acid) (PLGA), glycolide/trimethylene carbonate copolymers (PGA/TMC), poly-lactides (PLA), poly-L lactide (PLLA), poly-DL-lactide (PDLLA), L-lactide/DL-lactide copolymers, lactide/tetramethyl-glycolide copolymers, poly-caprolactone (PCL), poly-valerolacton (PVL), poly-hydroxy butyrate (PHB), poly vinyl alcohol (PVA) poly-hydroxyvalerate (PHV), polyvinylpyrrolidone (PVP), polyethyleneimine (PEI) and lactide/trimethylene carbonate copolymers, chitosan, carboxymethyl chitosan, chitin, pollulan, etc., or blends thereof.

In one embodiment, the first 101 polymer fiber is loaded with the therapeutic agents chosen from the group, but not limited to paclitaxel, rapamycin, cyclophosphamide, methotrexate, 5-fluorouracil, doxorubicin, cisplatin, hydroxyurea, leucovorin calcium, tamoxifen, flutamide, asparaginase, altretamine, mitotane, procarbazine hydrochloride, mechlorethamine, thioguanine, carmustine, lomustine, temozolomide, melphalan, chlorambucil, streptozocin, methotrexate, vincristine, bleomycin, vinblastine, vindesine, dactinomycin, 6-MP, daunorubicin, Lenalidomide, L-asparginase, doxorubicin, tamoxifen, antibiotics, antiseptic agents, anti-inflammatory drugs, such, ibuprofen, diclofenac, growth factors, phytochemicals such as curcumine, pipperlongumine, methyljasmonate, plumbagine, or combinations thereof.

In one embodiment, the second 102 polymer fiber is loaded with the therapeutic agents chosen from the group, but not limited to MGMT or AGT inhibitors like 06-Benzyl guanine, cell cycle/check point inhibitors like polo-like kinase (PLK) inhibitor (e.g. volasertib), cyclin dependent kinase (CDK) inhibitors (e.g. seliciclib, indirubin etc.,), topoisomerase inhibitors (e.g. adriamycin, camptothecin, etoposide, idarubicin, irinotecan, topotecan, mitoxantrone etc.), microtubule inhibitors (e.g. docetaxel, paclitaxel, vincristine etc.), antimetabolites (e.g. decitabine, gemcitabine, fludarabine etc.,), telomerase inhibitors, DNA and RNA replication inhibitors (e.g. clarithromycin, cytarabine, mitoxantrone HCl etc.) dihydrofolate reductase inhibitor, HDAC inhibitor, Bcl-2 and TNF-a inhibitors, PARP inhibitors, MAPK inhibitors, PI3K/Akt/mT0R inhibitors, integrase and protease inhibitors, Wnt/Hedgehog/Notch inhibitors, cAMP, lipide signaling inhibitors (e.g. PKC, PIM etc.), TGF-P inhibitors, tyrosine kinase inhibitors such as epidermal growth factor receptor (EGFR) inhibitors, vascular endothelial growth factor receptor (VEGFR) inhibitors, platelet derived growth factor receptor (PDGFR) inhibitors, fibroblast growth factor receptor (FGFR) inhibitors, Rous sarcoma oncogene/Breakpoint cluster region/Abl (Src-bcr-abl) inhibitors, Insulin-like growth factor 1 receptor (IGF-IR) inhibitors, FLT-3, HER-2, STATS, c-Kit, c-Met, ALK, ETA receptor inhibitor, HIF inhibitor, Syk inhibitor, Tie2 kinase inhibitor and the like), Vascular disrupting agents (e.g. plinabulin), antioxidant inhibitors like diethyl-dithiocarbamate, methoxyestradiol, 1-buthionine sulfoximine, 3-amino-1,2,4-triazole or the combinations thereof.

In one embodiment, the polymer fibers 101 and 102 have an average diameter between 1-50,000 nm. Polymer fibers 101 and 102 may be porous or non-porous, beaded or non-beaded, uniform or non-uniform, solid or hollow, or ribbon-shape in nature. In one embodiment, the first and second polymer fibers 101 and 102 possess different release kinetics. The drugs loaded in the fibers may be in their pure molecule form or in their slated form or in their nano-encapsulated form. In various aspects, the fibers 101 and 102 are randomly oriented fibers and/or aligned fibers.

Above all, the fibrous, flexible, biodegradable and biocompatible polymeric wafers, intended for the delivery of combination of therapeutic agents, for example anti-neoplastic drugs, locally to the diseased site in a controlled and sustained fashion. Moreover, the wafer consists of two or more kinds of electrospun fibers; each loaded with different drug molecules in such a way that the release kinetics of each fiber is optimal for the drugs loaded within and aids an optimal and combinatorial activity The optimum release kinetics is achieved by using two different polymers or blend of polymers or polymers with different molecular weight or polymers that have altered monomer ratio. Along with improving local bioavailability and sustained release of the drugs within, these drug delivery wafers can significantly reduce the systemic toxicities and associated adverse events.

The invention is further explained in the following examples, which however, are not to be construed to limit the scope of the invention as defined by the appended claims.

### EXAMPLES

### Example—1

In this example preparation of electrospun wafer loaded with DNA alkylating agent temozolomide (TMZ) and AGT inhibitor 06 Benzyl guanine (O6-BG) is described. In this wafer O6-BG is loaded in fibers of PLGA [poly (lactic-co-glycolic acid (50:50)] and TMZ in fibers of PLA (Poly lactic acid). For the effectiveness of TMZ-06-BG combinatorial therapy, O6-BG should be delivered prior to TMZ; and is the reason for its loading in PLGA (50:50). PLGA with faster degradation kinetics will release O6-BG loaded within it and TMZ will be released slowly from PLA fibers. PLGA solution in acetone premixed with 10% wt/wt O6-BG and PLA solution premixed with 20% wt/wt TMZ are taken in two different syringes and electrospun simultaneously at a rate of 3 ml/hr to a grounded metal surface. The tip to target distance was maintained as at 13 cm throughout the experiment.

The electrospray was carried out under ambient temperature, pressure and 55±5% humidity, by applying a potential of between 10-15 KV using a high voltage supply. The electrospun wafers were collected carefully and lyophilized for 96 hrs to remove any residual solvent and stored at low temperature, away from light and humidity.

### Example—2

In this example preparation of electrospun wafer loaded with (Carmustine) BCNU and O6 Benzyl guanine (O6-BG) is described. In this wafer O6-BG is loaded in fibers of PLGA [poly(lactic-co-glycolic acid (50:50)] and BCNU in fibers of PLA-PLGA (85:15) blend. PLA-PLGA(85:15) blend was prepared by dissolving the two polymers in acetone in 1:1 ratio and added with 20% wt/wt BCNU to it. 10% wt/wt 06-BG solution was prepared mixing the drug in PLGA(50:50) solution. The two different solutions were taken in two separate syringes and the electrospray was carried out in a sequential manner to get a final wafer consisting of intermittent layers loaded with the two drugs. In the first step the BCNU containing PLA-PLGA blend solution was electrosprayed using a potential of 13-14 KV at ambient temperature and pressure to a grounded metallic surface. After sufficient quantity of first layer formation, O6-BG containing PLGA(50:50) solution was electrosprayed on to the first layer at a potential of 10 KV. This process was repeated several times to get final wafer consisting of intermittent layers loaded with BCNU and OBG. The electrospun wafers were removed from the metallic surface and lyophilized for 96 h to remove any residual solvent and stored at low temperature, away from light and humidity.

### Example—3

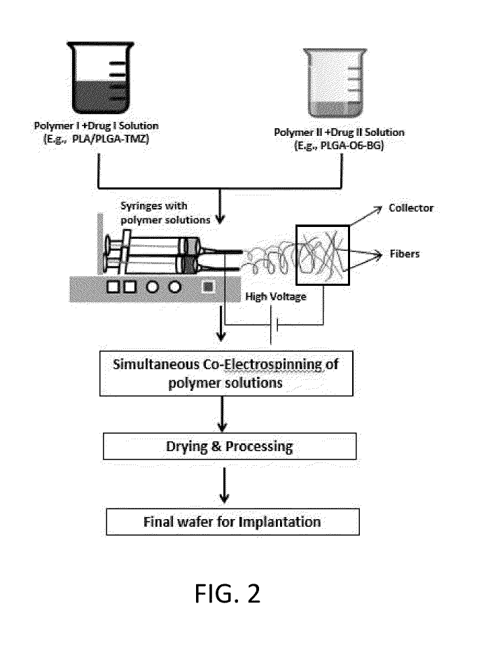

Referring to the schematic given in FIG. 2, for the preparation of flexible and biodegradable fibrous wafer, in step-i, polymer solution-I containing drug-I (e.g., PLGA (85:15)/PLA blend containing 20% wt/wt TMZ) and polymer solution-II containing drug-II(e.g., PLGA(50:50) containing 10% wt/wt O6-BG) are co-electrospun to yield polymeric wafers. The electrospun polymeric wafers thus formed are then lyophilized for 96 h to remove any residual solvent in it. The lyophilized wafers are then processed in aseptic conditions for desired shape and quantity.

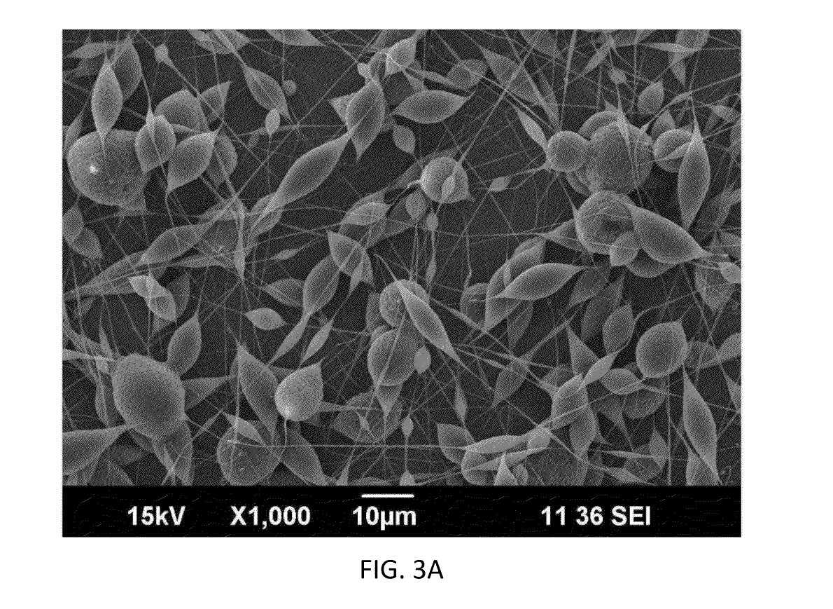

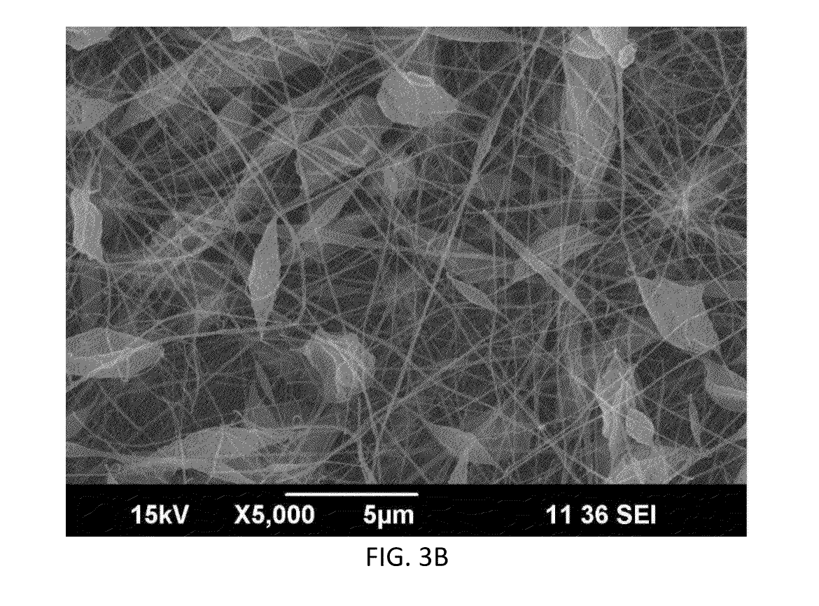

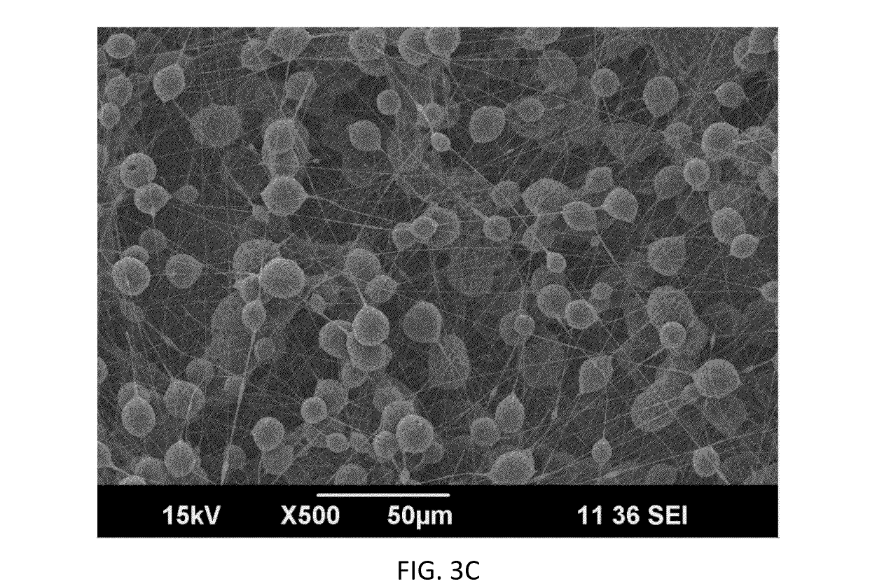

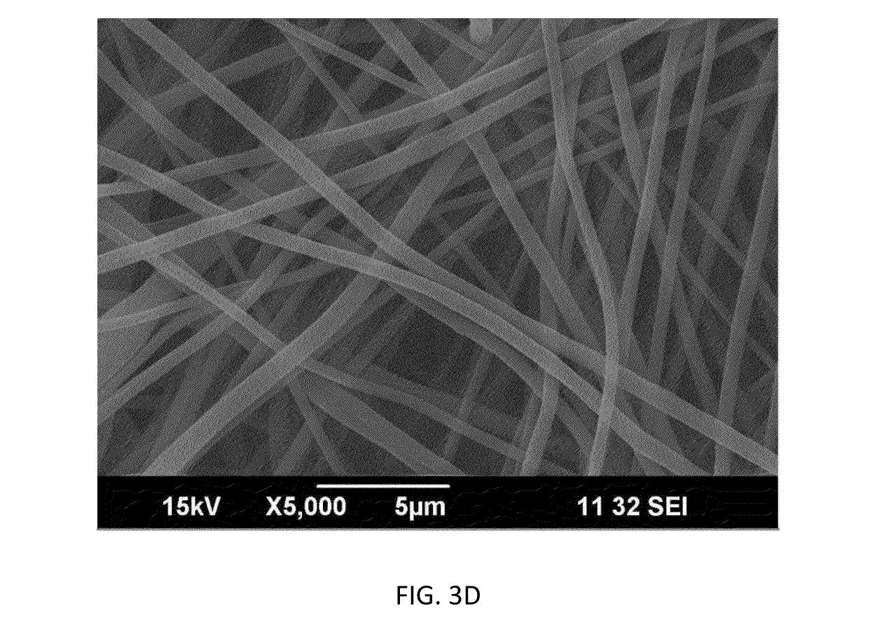

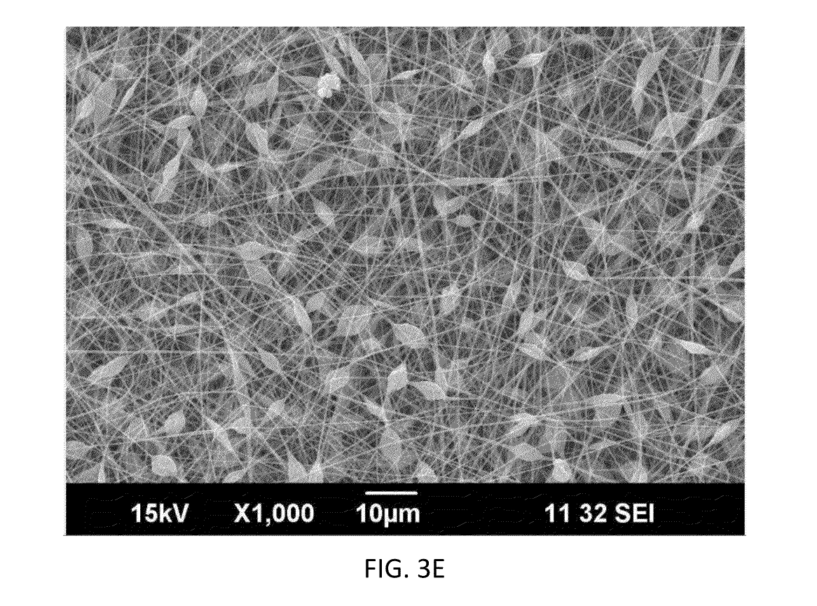

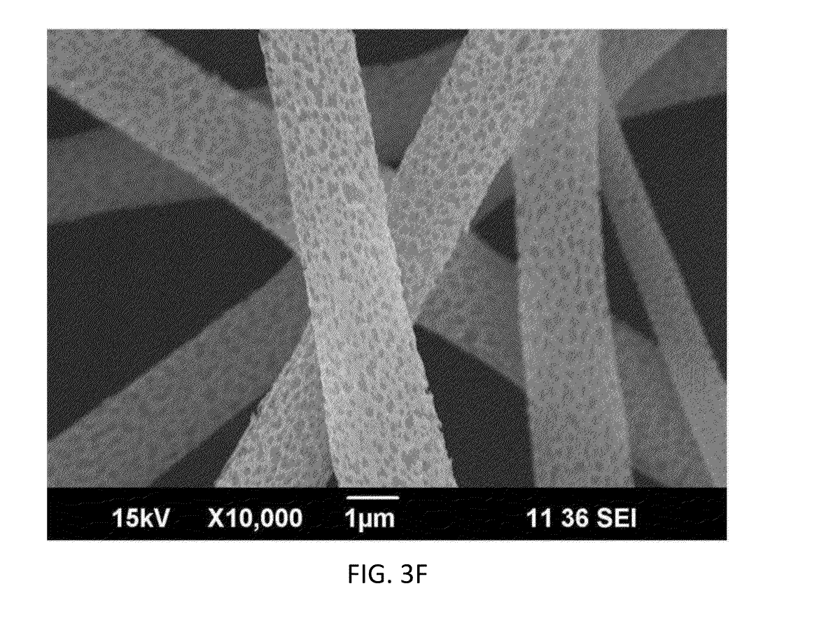

FIG. 3 shows different types of polymer fiber morphology that can be obtained during electrospinning or rotary-jet spinning The morphology can be threadlike, plain, ribbon type, beaded, porous etc. these fiber morphology will have profound effect on the drug release kinetics. For example, porous fibers will provide a burst and fast drug release as the porous nature will aid more solvent diffusion into the wafer and also by providing more surface area for drug elution.

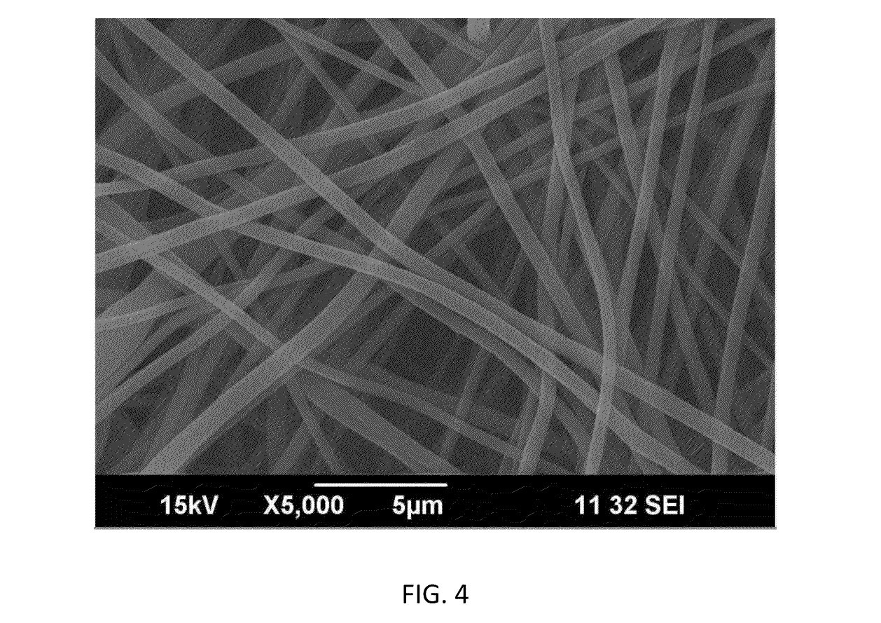

In relation to the above method of preparing embodiment, the polymeric fibers showed an average diameter of ˜2 mm as shown in FIG. 4. The fiber diameter can be varied from 10 mm to 50,000 nm depending on the polymer concentration, solvent, applied voltage, tip-target distance, etc., in the case of electrospun wafers.

### Example—3

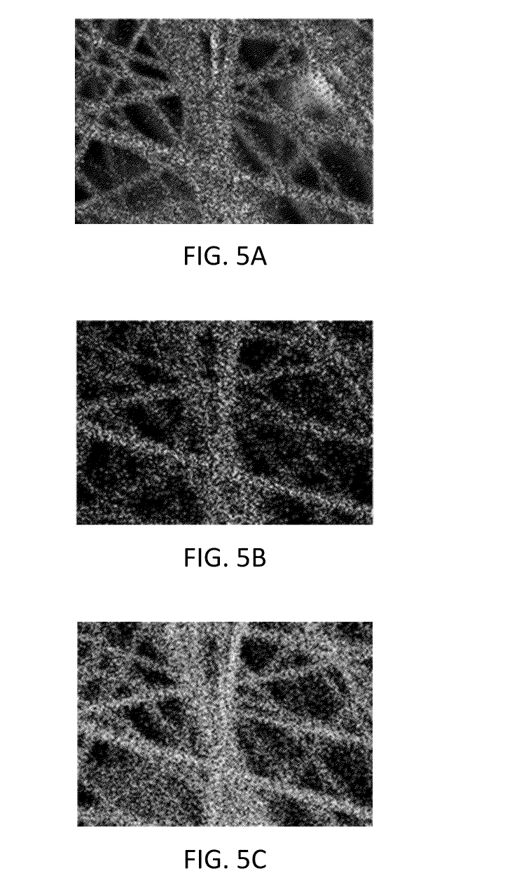

In yet another aspect of the above mentioned embodiment, the polymer fibers have shown uniform distribution of drugs throughout the fibers (FIGS. 5A-C). FIG. 5A depicts Temozolomid distribution. FIG. 5B depicts O6-BG distribution. FIG. 5C depicts merged distribution. Uniform drug distribution is considered very important for controlled drug release. existence of drug molecules as aggregates in fibers in a non-uniform nature will cause un-controlled drug release behavior

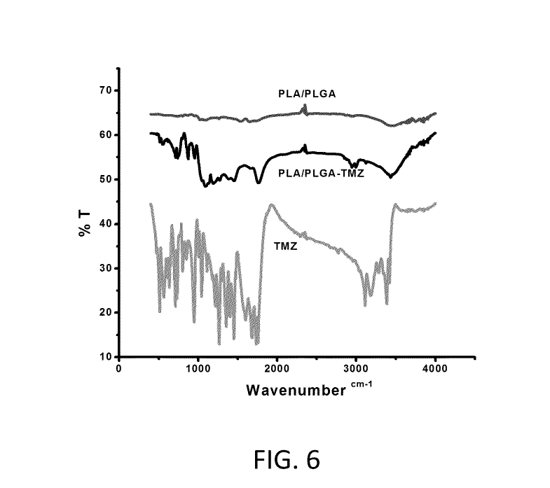

In yet another aspect of the above mentioned embodiment, the bare polymeric fibers (PLA-PLGA blend), drug loaded fibers (PLA-PLGA -TMZ) and pure drug (TMZ) shows distinct FTIR pattern as depicted in FIG. 6, shows the successful incorporation of the specific therapeutics in the nanomedicine construct. The incorporation and the effective drug loading will be depending on the interaction between the drug and the matrix forming material. For example, a drug having weak or no interaction toward the carrier polymer will mostly remain as separate entity on the voids of the electrospun wafers as aggregates and will cause burst release. But, on the other hand the drug having firm interaction towards its carrier molecule will be incorporated mostly throughout the fibers and will provide a much stable and extended release.

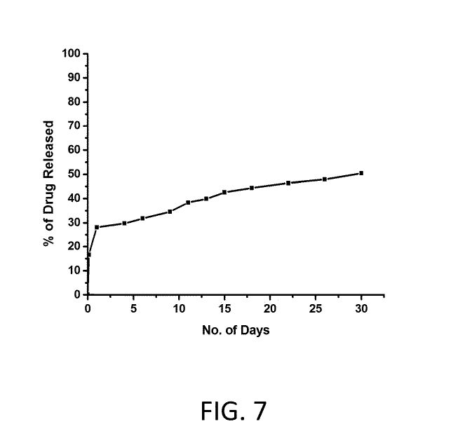

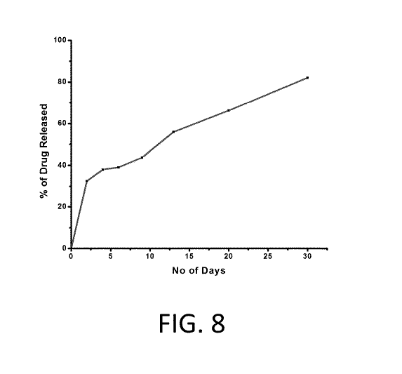

In yet another aspect of the same embodiment loaded with TMZ and O6-BG, both TMZ and O6-BG were released in a controlled and extended manner with near-zero order release kinetics as shown in FIG. 7 and FIG. 8 respectively. The wafer provided release for both drugs for more than 1 month. Since these wafers are implanted to the tumor resected cavity at the time of tumor removal, it is desirable that they provide maximum extended drug release.

### Example—4

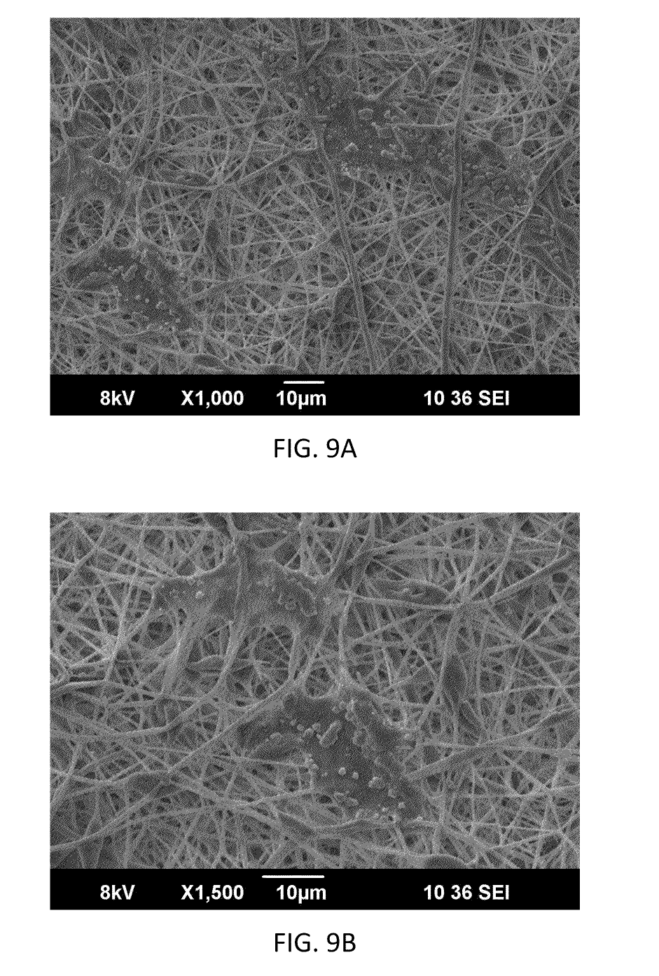

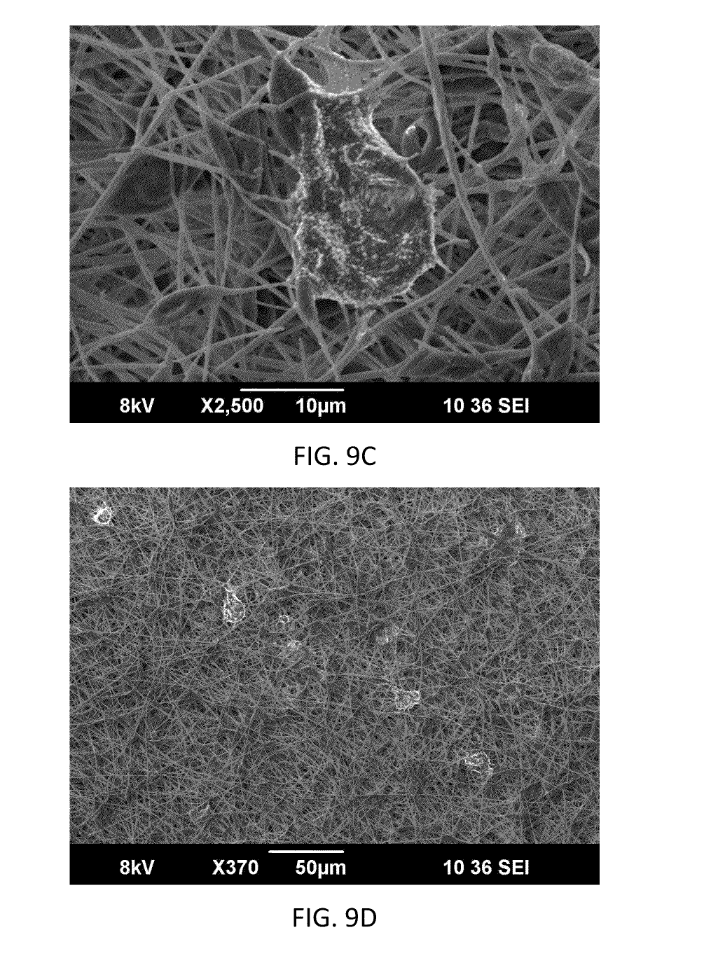

In yet another aspect of the same embodiment, the cell attachment studies on the wafer showed effective inhibition of cell attachment and cell growth by the drug loaded wafers. FIGS. 9A-D depict the SEM images results of cell attachment studies at different magnifications. FIGS. 9A-9B depict SEM images of cells attached to the bare wafer. The bare wafers aided attachment for the u87 mg glioma cells and the cells appeared in their normal stretched morphology. But, the drug loaded wafers effectively prevented any cell attachment and the cells were appeared small and round without proper attachment to the matrix, as depicted in FIGS. 9C-9D.

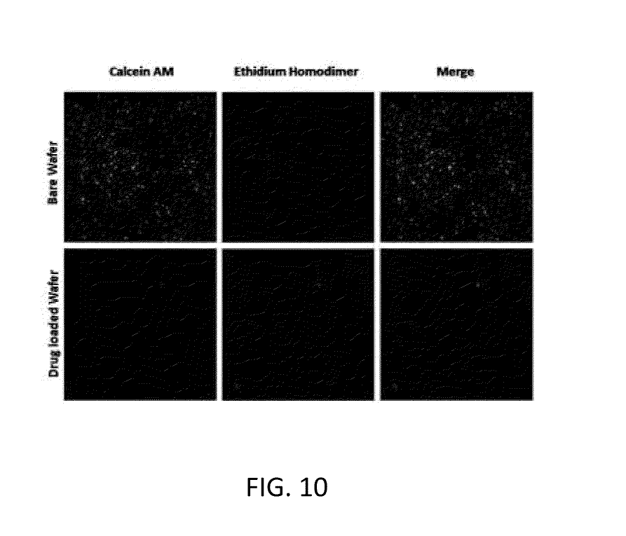

In yet another aspect of the above embodiment, the cell death induction by the drug delivery wafers is depicted in FIG. 10. Upper panel depicts the confocal microscopic images of live and attached cells on to bare wafer as seen by the green fluorescence due to the esterase activity in live cells; whereas the lower panel depicts the confocal microscopic images of the drug loaded wafers, where no cells were seen attached or proliferating. Bare PLA/PLGA wafers act as a supporting matrix for the cells to be attached, whereas the TMZ and O6-BG eluted from the drug loaded wafers prevent the cells from attaching into the matrix and inhibit the proliferating.

A flexible, biodegradable and biocompatible polymeric-fibrous drug delivery device is developed, in which different drugs for combination chemotherapy can be loaded in different kinds of polymer fibers having different degradation kinetics ultimately aiding controlled and sequential/simultaneous delivery of the drugs for enhance anticancer effects. The design of the nanomedicine is in such a way to simultaneously carry two different drugs and deliver it specifically and in a controlled fashion to the tumor cells in desired concentrations. The targeting is achieved by a specific biomarker ligand conjugated to the nanomedicine construct.

What is claimed is:

1. A flexible and biodegradable wafer system for delivering multiple therapeutic agents comprising:
first and second polymeric fibers; and
a plurality of therapeutic agents;
wherein the first and second polymeric fiber are configured as a flexible fibrous wafer loaded with the therapeutic agents, having mutually exclusive synergistic activity; and
wherein the fibrous wafer is configured to provide a combined therapy with sustained and controlled release of the therapeutic agents in the diseased site.

2. The system of claim 1, wherein the fibers are natural or synthetic biocompatible polymer at least one chosen from the group consisting of poly glycolic acid, poly(lactic-co-glycolic acid), glycolide/trimethylene carbonate copolymers, poly-lactides, poly-L-lactide, poly-DL-lactide, L-lactide/DL-lactide copolymers, lactide/tetramethyl-glycolide copolymers, poly-caprolactone, poly-valerolacton, poly-hydroxy butyrate, poly vinyl alcohol poly-hydroxyvalerate, polyvinylpyrrolidone, polyethyleneimine and lactide/trimethylene carbonate copolymers, chitosan, carboxymethyl chitosan, chitin, pollulan, and blends thereof.

3. The system of claim 1, wherein the first polymer fiber is loaded with the therapeutic agents chosen from the group consisting of paclitaxel, rapamycin, cyclophosphamide, methotrexate, 5-fluorouracil, doxorubicin, cisplatin, hydroxyurea, leucovorin calcium, tamoxifen, flutamide, asparaginase, altretamine, mitotane, procarbazine hydrochloride, mechlorethamine, thioguanine, carmustine, lomustine, temozolomide, melphalan, chlorambucil, streptozocin, methotrexate, vincristine, bleomycin, vinblastine, vindesine, dactinomycin, 6-MP, daunorubicin, Lenalidomide, L-asparginase, doxorubicin, tamoxifen, antibiotics, antiseptic agents, anti-inflammatory drugs, growth factors, curcumine, pipperlongumine, methyljasmonate, plumbagine, and combinations thereof.

4. The system of claim 1, wherein the second polymer fiber is loaded with the therapeutic agents chosen from the group consisting of MGMT or AGT inhibitors, cell cycle/check point inhibitors, cyclin dependent kinase inhibitors, topoisomerase inhibitors, microtubule inhibitors, antimetabolites, telomerase inhibitors, DNA replication inhibitors, RNA replication inhibitors, dihydrofolate reductase inhibitor, HDAC inhibitor, Bcl-2 and TNF-a inhibitors, PARP inhibitors, MAPK inhibitors, PI3K/Akt/mT0R inhibitors, integrase inhibitors, protease inhibitors, Wnt/Hedgehog/Notch inhibitors, cAMP, lipide signaling inhibitors, TGF-P inhibitors, tyrosine kinase inhibitors, epidermal growth factor receptor inhibitors, vascular endothelial growth factor receptor inhibitors, platelet derived growth factor receptor inhibitors, fibroblast growth factor receptor inhibitors, Rous sarcoma oncogene/Breakpoint cluster region/Abl inhibitors, insulin-like growth factor 1 receptor inhibitors, FLT-3 inhibitors, HER-2 inhibitors, STATS inhibitors, c-Kit inhibitors, c-Met inhibitors, ALK inhibitors, ETA receptor inhibitor, HIF inhibitor, Syk inhibitor, Tie2 kinase inhibitor, Vascular disrupting agents, antioxidant inhibitors, and the combinations thereof.

5. The system of claim 1, wherein the polymer fibers have an average diameter between 1-50,000 nm.

6. The system of claim 1, wherein the polymer fibers are porous.

7. The system of claim 1, wherein the polymer fibers are non-porous.

8. The system of claim 1, wherein the polymer fibers are beaded.

9. The system of claim 1, wherein the polymer fibers are non-beaded.

10. The system of claim 1, wherein the polymer fibers are uniform.

11. The system of claim 1, wherein the polymer fibers are non-uniform.

12. The system of claim 1, wherein the polymer fibers are solid.

13. The system of claim 1, wherein the polymer fibers are hollow.

14. The system of claim 1, wherein the polymer fibers are ribbon-shape in nature.

15. The system of claim 1, wherein the first and second polymer fibers possess different release kinetics.

16. The system of claim 1, wherein the drugs loaded in the fibers are in their pure molecule form.

17. The system of claim 1, wherein the drugs loaded in the fibers are in their slated form.

18. The system of claim 1, wherein the drugs loaded in the fibers are in their nano-encapsulated form.

19. The system of claim 1, wherein the fibers are randomly oriented fibers.

20. The system of claim 1, wherein the fibers are aligned fibers.
